(19) United States
(12) Patent Application Publication
(10) Pub. No.: US 2022/0023492 A1
(43) Pub. Date: Jan. 27, 2022

(54) GEL, IN PARTICULAR FOR USE IN A WOUND TREATMENT AGENT

(71) Applicant: SANIXTREME GMBH & CO KG, Frankfurt am Main (DE)

(72) Inventor: Marco Hess, Neu-Isenburg (DE)

(73) Assignee: SANIXTREME GMBH & CO KG, Frankfurt am Main (DE)

(21) Appl. No.: 17/282,999

(22) PCT Filed: Oct. 4, 2019
(86) PCT No.: PCT/EP2019/000286

§ 371 Date: Apr. 5, 2021

(30) Foreign Application Priority Data
Oct. 5, 2018 (DE) 102018007857.5

(51) Int. Cl. A61L 26/00

(52) U.S. Cl. CPC A61L 26/0066; A61L 26/0004; A61L 2300/442; A61L 2300/106; A61L 2300/404; A61L 2300/802

(57) ABSTRACT

A gel is to be disclosed, in particular for use in a wound treatment composition, with which a particularly high storage stability and long-term stability can be achieved with high microbiocidal activity. For this purpose, the gel according to the invention comprises an inorganic silicate as thickener and an electrochemically activated starting saline solution which has a free chlorine content of more than 300 mg/l. In addition, a colored gel having the properties described above can be obtained by adding suitable pigments.

The invention relates to a gel, in particular for use in a wound treatment agent, disinfection of drinking water, on mucous membranes, highly perfused skin regions, for example also sexual organs, or other applications for human or animal use.

By electrochemical activation, in particular by electrolysis of sodium chloride solutions, electrochemically activated salt solution (also referred to as “anolyte”) can be produced, which is a highly effective disinfectant with exceptional microbiocidal properties. Such anolyte can be used, for example, depending on the chosen dilution, in applications for surface disinfection, e.g. of workplaces, tables, floors, but also for cold disinfection actions, in agriculture for the elimination of microbial organisms, for laundry, in applications for swimming pools or even as prophylaxis against athlete's foot. However, if necessary and in a suitable formulation, such anolyte can also be used as an active ingredient to combat microbes or germs of any kind, in particular bacteria, viruses, fungi or the like.

Among other things, it is also desirable to use such an anolyte or such an electrochemically activated salt solution, which has, among other things, a free chlorine content of more than 300 mg/l, as an agent for treating wounds, in particular for cleaning and disinfecting wounds to accelerate the healing process. For this purpose, or also generally to expand the application possibilities, it is desirable to provide the anolyte or the electrochemically activated salt solution in a form which, on the one hand, permits comparatively simple handling, especially when applied to the wounds to be treated, and, on the other hand, also permits a high microbiocidal efficacy of the active ingredient with a particularly long shelf life, i.e. in particular high long-term stability. To this end, the aim is to keep the electrochemically activated salt solution, i.e. the anolyte, in the form of a gel which, in addition to the conditions mentioned, ensures particularly good storage stability and long-term stability, especially with regard to the microbiocidal activity of the anolyte.

The invention is therefore based on the task of providing a gel, in particular for use in a wound treatment agent or as a wound treatment agent, which meets the above requirements in a particular way. In addition, a particularly suitable process for the production of the gel is to be disclosed.

With regard to the gel, this task in accordance with the invention is solved in that the gel comprises an electrochemically activated saline solution which has a free chlorine content of more than 300 mg/l and, as thickener, an inorganic layered silicate in whose layers hydroxide anions are incorporated.

Advantageous embodiments of the invention are the subject of the subclaims.

Surprisingly, it has been found that among the plurality of possible thickeners or gel formers, the desired combination of properties for the gel is achievable by selecting a suitably configured inorganic silicate as thickener. By selecting such a material as thickener, in particular the otherwise desired properties of the electrochemically activated saline solution, especially the microbiocidal activity, can be maintained even after the gelation process.

A particularly advantageous gel former or thickener is the layered silicate available under the brand name “Laponite”. This has the structure of a layered silicate. The distance between two adjacent layers is about one nanometer. Hydroxide and possibly also fluorine anions are incorporated in the layers. The corresponding cation is magnesium. Part of the magnesium is replaced by lithium; as a result, some sites intended for magnesium remain unoccupied in the molecule. This results in a negative charge, which is replaced by exchangeable ions—mostly sodium ions. In this way, the individual layers are kept together and in equilibrium. The sodium ions are located between the layers, i.e. not in the molecular lattice.

When Laponite® is dispersed in water, the exchangeable cations are dissolved out. As a result, the remaining negatively charged layers repel each other. By adding an electrolyte, however, this negative charge is reduced again to such an extent that a bond between cut surfaces and the weakly positively charged platelet edges is preferentially formed. The platelets connected by surfaces and edges form the shape of a house of cards. The gel structure collapses easily under heavy loading, but rebuilds quickly when standing. Surprisingly, the choice of this thickener material makes it possible to achieve particularly homogeneous and stable gelation of an electrochemically activated saline solution.

To ensure the desired high microbiocidal activity of the gel, the starting saline solution advantageously has a free chlorine content of more than 500 mg/l, particularly preferably more than 600 mg/l, and/or a redox potential of between 600 and 1000 mV, particularly preferably between 700 and 900 mV, especially about 800 mV. In an alternative or additional advantageous embodiment, the starting saline solution, i.e. particularly preferably the anolyte, has a conductivity of between 12 and 16 mS/cm, particularly advantageously of about 14 mS/cm, and/or a pH of between 6 and 8, particularly preferably of about 7.

The inorganic silicate intended for use as a thickener or gelling agent is, especially in combination with the particularly preferably selected anolyte, in an advantageous embodiment a lithium magnesium sodium silicate. The particularly preferred Laponite RD® or Laponite RDS® is available as a lithium magnesium sodium silicate, for example as a mixture of the materials SiO₂ (preferably about 54.5-59.5%), MgO (preferably about 26-27.5%), Na₂O (preferably about 2.8-5.6%) and Li2O (preferably about 0.8%). Advantageously, P₂O₅ (preferably about 4.4%) can be provided in addition. This gelling agent is particularly preferred because of its swelling capacity at low shear forces and is particularly suitable for combination with the electrochemically activated saline solution, since it cannot be oxidized further and thus does not impair the effectiveness and reactivity of the saline solution. In addition, it is usually produced synthetically and is therefore particularly efficient due to its freedom from impurities. However, other water-absorbing gelling agents, in particular those from the montmorillonite group, may also be suitable.

For the production of the gel, in particular using the above-mentioned electrochemically activated saline solution in combination with Laponite as thickener or gel former, the mixing ratio thickener/saline solution can be suitably selected according to the desired application, i.e. the desired viscosity. For comparatively viscous applications that are not intended to drip, a mixing ratio of thickener/boiling salt solution of between about 8 and about 25 percent by weight (thickener content), particularly preferably of about 8 percent by weight, is preferably selected. For low-viscosity gels, which are to flow into a wound or the like, for example, a mixing ratio of between 4 and 8 percent by weight (thickener) can preferably be selected. For particularly low-viscosity preferred applications, for example for hand and surface disinfection, on the other hand, a mixing ratio of between 1 and 4 percent by weight (thickener content) is preferably selected. Particularly preferably, the gel is produced using the above-mentioned components in such a way that it is particularly easy to handle for the intended use, i.e. in particular as a wound treatment agent or as a component of a wound treatment agent. For this purpose, it is advantageously intended, among other things, to produce the gel in such a way that it has a viscosity of between 4500 and 6500 mPas, particularly preferably of about 5500 mPas.

Surprisingly, the gel produced is also particularly suitable for high long-term stability and storage stability while retaining the high microbiocidal activity of the starting saline solution, in particular the anolyte that is preferably used. The combination of the starting saline solution with the inorganic silicate selected as thickener apparently allows particularly favorable incorporation of the active ingredient into the thickener so that, in the manner of encapsulation of the active ingredient, an exchange of substances with the environment, which could promote decomposition of the active ingredient, is prevented. By incorporating the electrochemically activated starting saline solution into the gel, a particularly favorable stabilizing effect for the active ingredient can thus be achieved.

Surprisingly, it has also been found that the pH value of the gel produced is of particular importance for the desired high long-term stability and storage stability of the gel. Therefore, it is intended to adjust the pH value of the gel appropriately with regard to the ingredients used. For the particularly preferred ingredients, namely in particular anolyte in combination with laponite as thickener or gel former, a pH value of at least 8, particularly preferably of about 9, is particularly suitable for a particularly high long-term stability. Advantageously, therefore, a pH of about 9 is set for the gel during production of the gel, in particular by suitable selection of the mixing ratio of thickener to saline solution.

With regard to the process, the above-mentioned task is solved by adding an inorganic silicate as a thickener to electrochemically activated starting saline solution which has a chlorine content of more than 300 mg/l. The thickener is an inorganic silicate.

Preferably, a lithium magnesium sodium silicate, preferably a laponite, is added as the inorganic silicate.

In a particularly advantageous embodiment, and in particular in order to ensure the desired high long-term stability and storability of the gel while maintaining a high microbiocidal efficacy, the thickener or the gel former is admixed in such a way that the resulting gel has a pH of at least 8, preferably 9.

The advantages achieved with the invention consist in particular in the fact that, based on an electrochemically activated starting saline solution with a free chlorine content of more than 300 mg/l, a gel suitable for use as a wound treatment agent with a particularly high microbiocidal effect can be reliably provided by adding an inorganic silicate as a thickener or gel former. When used as a wound treatment agent, the gel also exhibits a cooling effect that is perceived as particularly pleasant. The particularly preferred setting of the pH value of the gel to a value of about 9, in particular by suitable selection of the mixing ratios, makes it possible to achieve a particularly high long-term stability and shelf life of the gel without excessive undesirable loss of microbiocidal activity. Particularly in combination with Laponite, a gel can be provided that is stable in storage and shape while retaining its microbiocidal activity and retains its consistency without tending to run or melt.

An embodiment example of the invention, in particular concerning the preparation of the gel, is explained in more detail below.

In the present embodiment example, the electrochemically activated salt solution obtainable by electrolysis of sodium chloride solutions is used as the base material or starting saline solution. Such an anolyte has a free chlorine content of about 700 mg/l, a redox potential of about 800 mV, a pH of about 7 and an electrical conductivity of about 14 mS/cm.

Laponite®, an inorganic silicate, is added to this electrochemically activated starting saline solution as a thickener or gelling agent. The thickener is added at a mixing ratio of thickener to saline solution of about 8%, with the viscosity of the resulting gel being determined as one of the process control parameters. This is advantageously set to a target value of about 5500 mPa. Furthermore, the pH value of the gel is determined as a parameter of particular importance for the long-term stability and storability of the resulting gel. The thickener is added in such a way that the resulting gel has a pH of at least 8, preferably about 9.

The thickener or gelling agent is added by successive sprinkling into the mixture while stirring. This is followed by pre-swelling while stirring for a long time and then homogenization while increasing the stirring speed.

Furthermore, it has been possible to convert this gelatinized form into a colored form so that it can be used in human and veterinary medicine. The coloring of the otherwise colorless gel is intended to enable coverage control.

Surprisingly, it turned out that among the large number of possible pigments, only a very limited number are compatible with the electrochemically activated water or in combination with the thickener or gel former. The compatible pigments are Symrise Green C.I. 74260, Merck Rona Colorona Majestic Green, Merck Rona Tim iron Splendid Blue, Clariant Cosmenyl Green 2 GLS gran, Clariant Sanolin Yellow BG30, Kremer Pigments Ultramarine Dark, Indigo Trisulfonic Acid Tripotassium Salt, Bayferrox 110M, Pigment Red 101, Bayferrox 420 Pigment Yellow 042, Cabot Elftex 430 Carbon Black, Degussa Gas Carbon Black S160, Evonik Aeroxide TiO2 P25 fumed. The combination of electrochemically activated water and/or said thickener with one or more of these or similar pigments is considered independently inventive.

The pigments Ulttramarin dark, Bayferrox 110M and Bayferrox 420 are particularly suitable for use in gels based on the starting saline solution containing more than 300 mg/l free chlorine. All pigments produce very good color impressions already at a concentration of 0.1 wt. %. The concentration can also be increased to above a concentration of 2 wt. % to obtain a stronger color appearance. By selecting these mentioned pigments, the desired properties of the electrochemically activated saline, especially the microbiocidal activity, can be obtained.

To prepare the colored gel, 0.1 wt % to 2 wt %, preferably 1 wt %, of the desired pigment is first added to the starting saline solution. Then, the manufacturing process described above is carried out with the thickener or gel former.

Surprisingly, it has also been found that the colored gels produced are particularly suitable for high long-term stability and storage stability while retaining the high microbiocidal activity of the starting saline solution.

1. Use of an electrochemically activated saline solution which has a free chlorine content of more than 300 mg/l and a redox potential of between 600 and 1000 mV, preferably between 700 and 900 mV, particularly preferably of about 800 mV, for the production of a gel which comprises, as thickener, an inorganic layered silicate in whose layers hydroxide anions are incorporated.

2. Use according to claim 1, wherein the starting saline solution has a pH between 6 and 8, preferably of about 7.

3. Use according to claim 1 or 2, wherein the starting saline solution has a free chlorine content of more than 500 mg/l, preferably of more than 600 mg/l.

4. Gel obtained by the use according to any one of claims 1 to 3.

5. Gel according to claim 4, wherein the mixing ratio thickener/saline solution is between about 1 and about 25% by weight.

6. Gel according to claim 4 or 5, wherein as thickener is provided a lithium magnesium sodium silicate, particularly preferably Laponite®.

7. Gel according to any one of claims 4 to 6, having a pH of at least 8, preferably of 9, and/or a viscosity of between 4500 and 6500 mPa, preferably of about 5500 mPa.

8. Colored gel according to any one of claims 4 to 7, wherein the mixing ratio pigment/saline solution is between about 0.1 and about 2% by weight.

9. The colored gel of claim 8, wherein the pigment is one or more of Symrise Green C.I. 74260, Merck Rona Colorona Majestic Green, Merck Rona Tim iron Splendid Blue, Clariant Cosmenyl Green 2 GLS gran, Clariant Sanolin Yellow BG30, Kremer Pigments Ultramarine Dark, Indigo Trisulfonic Acid Tripotassium Salt, Bayferrox 110M, Pigment Red 101, Bayferrox 420 Pigment Yellow 042, Cabot Elftex 430 Carbon Black, Degussa Gas Carbon Black S160, and/or Evonik Aeroxide TiO2 P25 fumed is provided.

10. Use of a gel according to any one of claims 4 to 9 in a wound treatment composition.
